(19) United States
(12) Patent Application Publication
(10) Pub. No.: US 2016/0061812 A1
(43) Pub. Date: Mar. 3, 2016

(54) Diagnosis and Treatment of Arthritic Conditions

(71) Applicant: THE UNIVERSITY OF BIRMINGHAM, Birmingham (GB)

(72) Inventors: Stephen Peter Young, Birmingham (GB); Karim Raza, Birmingham (GB); Sabrina Reenu Raizada, Wolverhampton (GB)

(21) Appl. No.: 14/774,622

(22) PCT Filed: May 1, 2013
(86) PCT No.: PCT/GB2013/051122

§ 371 Date: Sep. 10, 2015

Related U.S. Application Data
(60) Provisional application No. 61/787,491, filed on Mar. 15, 2013.

(51) Int. Cl. G01N 33/493; C07K 14/525; C07K 16/24

(52) U.S. Cl. CPC G01N 33/493; C07K 16/241; C07K 14/525; G01N 2800/52; G01N 2570/00; G01N 2800/102; A61K 2039/505

(57) ABSTRACT

Provided is a method of determining the likelihood that a patient, with a disorder treatable with a TNF antagonist, will respond to administration of a TNF-antagonist. The method comprises determining the likelihood of the patient's response to said antagonist based on a metabolic profile of a urine sample from said patient. Methods of treatment and kits for use in said methods are also provided.

### FIELD OF THE INVENTION

The present invention provides a method of determining the likelihood that a patient, with a disorder treatable with a TNF antagonist, will respond to administration of a TNF-antagonist, and methods of treatment and kits for use in said methods.

### BACKGROUND TO THE INVENTION

The introduction of anti-TNFα treatment has revolutionised the management of Rheumatoid Arthritis RA (1-4). Several agents are available within this class but response rates are imperfect: only 26-42% of patients achieve a good EULAR response within 6 months (5-7). Given the high cost of these therapies, and implications for disease progression in non-responders waiting for 3 to 6 months for clinical reassessment, the ability to predict treatment responses at baseline is an important goal.

The aetiology of RA is not fully understood but involves both genetic and environmental factors. In addition to synovitis there are widespread systemic effects mediated by proinflammatory cytokines that impact upon metabolism. Surprisingly, we have found that metabolic profiling predicts the response to anti-TNFα therapy in patients with rheumatoid arthritis. There were clear differences in the metabolic profiles of baseline urine samples of patients with RA who responded well to anti-TNF therapy compared with those who did not.

This difference may be important as a novel predictor of responses to TNF antagonists. It is envisaged that this approach can also be applied to a range of similar conditions as well, including and Psoriatic Arthritis (PsA), Lupus and AS (Ankylosing spondylitis).

### SUMMARY OF THE INVENTION

Thus, in a first aspect, the present invention provides a method of determining the likelihood that a patient, with a disorder treatable with a TNF antagonist, will respond to administration of a TNF-antagonist, the method comprising determining the likelihood of the patient's response to said antagonist based on a metabolic profile of a biological fluid sample from said patient.

The metabolic profile may be determined by assaying for the presence of one or more metabolites in the sample from the patient. The sample is ideally a baseline sample, i.e. taken before treatment has been initiated, especially if said treatment is with a TNF antagonist. The sample is a biological fluid. In some embodiments, this may be serum. In some embodiments, this may be urine. For the sake of simplicity, reference herein is predominantly made to urine as the sample, but this may also include serum or other biological fluids unless otherwise apparent.

The metabolites may be any metabolites found in the sample and associated with responsiveness of the patient to a TNF antagonist. In some embodiments, the metabolite is a metabolite associated with tissue degradation or a catabolic process. In some embodiments, the metabolite to be profiled is histamine. In some embodiments, the metabolites to be profiled are any one, and ideally all, of: histamine, glutamine, xanthurenic acid and/or ethanolamine. In some embodiments, the metabolites to be profiled are any one, and ideally all, of: p-hydroxyphenylpyruvic acid, phosphocreatine, thymine, creatinine, phenylacetic, acid and/or xanthine may also be assayed/profiled. This may be in addition or in place of histamine, glutamine, xanthurenic acid and/or ethanolamine.

The metabolic profile (or fingerprint or metabolomic data) may comprise of merely the presence or absence of a particular metabolite, but may also be formed from the presence or absence of several metabolites. Furthermore, the levels of a certain metabolite or certain metabolites may be determined and this may be indicative of responsiveness to a TNF antagonist. For example, certain metabolites may be upregulated, whilst other may be downregulated in responsive patients. Upregulation or downregulation may be compared to a reference or threshold value. The profile may, therefore consist of one or more piece of data for each metabolite. These may be, for instance: presence/absence; level; and/or level above/below a threshold value. Thus, a profile can be built up based on the presence, absence or levels of a certain metabolite or mixtures of two or more metabolites. In some embodiments, the profile may be a mixture of upregulated metabolites; a mixture of downregulated metabolites: or a mixture of upregulated metabolites and downregulated metabolites. Suitable assays for determining the presence, absence or levels of a metabolite in urine are well known in the art, but may include HPLC, for instance, in patients with RA, for example, who are likely to respond to TNF antagonists, there may high levels of glutamine; phenylacetic acid and/or histamine. This is typically in the baseline urine samples. Higher levels of methylamine and/or creatinine in the urine post anti-TNF therapy may also be seen, so these may be determined separately post-treatment. Similar changes in metabolites may also be seen in the urine samples of patients with PsA who are likely to respond to TNF antagonists.

In any patient, threshold levels of the metabolite may be outside the normal 95% reference interval for that metabolite, indicating a higher or lower level of that metabolite.

Thus, a suitable data set can be built up (as a fingerprint). This may then be overlaid matched or compared with a reference fingerprint to thereby determine the likelihood that the patient is a responder or non=responder to TNF antagonists. The reference fingerprint may have the same or more data points (i.e. metabolites) compared to the patient's fingerprint. It is also envisaged that a fingerprint for a patient may be built up as part of the present methods. It may continue to be developed over time. This may be compared to a reference fingerprint or may compared against previous fingerprints from the same patient to determine changes over time and thus allow the progression of the disease to be tracked.

In RA patients, threshold levels of phenylacetic acid would typically be above the normal 95% reference interval of 0.364 μg/mg (micrograms per milligram) creatinine. In RA patients levels of xanthurenic acid would typically be above the normal 95% reference interval of 1.18 μg/mg (micrograms per milligram) creatinine. These figures come from Biomedical chromatography (2008) v22 pp 1346-1353.

We have measured (using classical biochemistry approaches) both glutamine and xanthurenic acid. These correlated with the peaks in the NMR spectra. As an example, the mean xanthurenic acid concentration was 2.53 micro molar in urine of responding patients and 2.15 micro molar in non-responding patients (so 18% more in responders). The respective values for glutamine were 336 μM and 290 μM (16% more).

Responsiveness as referred to herein relates to the clinical outcome of administering a TNF antagonist. This may be whether or not the patient, if prescribed or administered a TNF antagonist would benefit, for instance at least in the sense of an amelioration of symptoms or slowing of the progression of the disorder. Side effects may be discounted in the assessment of responsiveness. Criteria for assessing this are known in the art and examples are also mentioned herein. Non responsiveness may be considered to be the opposite or responsiveness—i.e. no change or even a worsening of symptoms.

Disorders that may be treated using a TNF inhibitor include rheumatoid arthritis (RA), ankylosing spondylitis (AS), Crohn's disease, psoriasis and its associated arthritis (PsA), hidradenitis suppurativa and/or refractory asthma. In some embodiments the disorder may be RA. In other embodiments, the disorder may be AS or psoriasis (and PsA). In some embodiments, Behcets Disease may also be included.

The TNF antagonist may be considered to be a TNF inhibitor and may include anti-TNF antibodies, especially monoclonal antibodies. Examples of these are known in the art and approved products include etanercept (Enbrel), infliximab (Remicade), adalimumab (Humira), certolizimumab pegol (Cimzia) and golimumab (Simponi). Etanercept and/or infliximab are preferred. The TNF antagonist for treating the disorder may not necessarily be the same as the TNF antagonist that may be used to treat the patient, if the use of a TNF antagonist is appropriate, but in general the two are interchangeable. Reference herein to a single TNF antagonist may be considered to relate to one or more TNF antagonists unless otherwise apparent. The TNF is ideally TNF-alpha.

It will be appreciated that definitive answers, although ideal, may not always be possible, hence the likelihood referred to herein. Generally, the best that can be done is that a percentage range is provided: say there is at least a 50-70% or 60-80% or at least 70-90% or at least 75-85% or at least 80-90% or at least 85-95% or at least 90-95% or more chance that the patient will be responsive to a TNF antagonist.

In a further aspect, the invention provides a method of determining whether a patient with RA will respond to administration of a TNF inhibitor, comprising assaying metabolites in a biological fluid sample from the patient and correlating changes in metabolite levels with a likelihood of a positive response to said inhibitor.

In a still further aspect, the invention provides a screening method comprising determining the likelihood that two or more patients, with a disorder treatable with a TNF antagonist, will respond to administration of a TNF-antagonist, the method further comprising determining the likelihood of the patients' response to said antagonist based on a metabolic profile of biological fluid samples from each patient. This may also be considered to be a method of identifying patients, for instance in a certain population, that have the disorder and may respond to a TNF antagonist.

In a further aspect, the invention provides a method for the treatment or prophylaxis of a disorder treatable with a TNF antagonist, comprising: identifying that the patient will respond to administration of a TNF-antagonist by determining the likelihood of the patient's response to said antagonist based on a metabolic profile of a biological fluid sample from said patient; and administering a suitable treatment to said patient. The suitable treatment may comprise a TNF antagonist if the patient is determined to be likely to respond to a TNF antagonist.

Alternatively, it may not comprise a TNF antagonist if the patient is determined to be unlikely to respond to a TNF antagonist. Treatments other than TNF antagonists are well known for the disorder. For instance, they may include NSAIDs for RA.

Infliximab and etanercept were found to alter metabolites in the urine differently. For instance, there were clear differences in the metabolites at 12 weeks post treatment. Increases in the metabolites hippuric acid, citrate and/or lactic acid, and ideally all of them, may be associated with infliximab treatment. Increases in the metabolites choline, phenylacetic acid, urea, creatine and/or methylamine, and ideally all of them, were associated with etanercept treatment. Also provided is a method for determining the likelihood that a patient has been treated with Infliximab or etanercept, the method comprising determining the likelihood based on a metabolic profile of a urine sample from said patient, wherein increases in the metabolites hippuric acid, citrate and/or lactic acid, and ideally all of them, may be associated with infliximab treatment and increases in the metabolites choline, phenylacetic acid, urea, creatine and/or methylamine, and ideally all of them, were associated with etanercept treatment. The increases may be compared against a baseline, i.e. pre-treatment.

For RA, infliximab and/or etanercept are particularly preferred as the TNF antagonist. For Crohn's disease, infliximab is particularly preferred as the TNF antagonist. Infliximab and etanercept currently are both used in treating RA, but only infliximab is used in treating Crohn's disease. So analysis of urines of Crohn's disease may show differences with RA.

In a further aspect, the invention provides a kit for determining the metabolic profile of a biological fluid sample. The kit is suitable for use in the present methods. It may comprise: a set of at least one control metabolite, but preferably two, three, four or more control metabolites; and means for collecting and/or testing samples and the metabolites therein. Suitable means for collecting serum are known as those for collecting urine, which may include pots or vials, for instance. The control metabolites correspond to the metabolites being tested (assayed) for in the sample from the patient.

Provides is, therefore, a kit for determining the metabolic profile of a biological fluid sample comprising: means for collecting and/or testing samples and the metabolites therein; and a set of at least one control metabolite corresponding to the metabolites being tested for in the sample from a patient.

Thus, the control metabolites may include histamine and so forth as described herein. In other words, a kit might include a set of standard metabolites which can be used to confirm the presence of the key metabolites. These may be also present at a range of concentrations for use in Mass spectrometric (MS) on NMR analysis of urines. Such standards would also be useful in ELISA (enzyme linked immune sorbent assays) in which antibodies specific to each of the metabolites could be used to assess the concentration of the individual metabolites and so derive the overall metabolite fingerprint. Indeed, the kit itself may also comprise an ELISA kit (sub-kit) comprising one or antibodies specific each metabolite to be detected. The ELISA kit would also comprise means for detecting said antibodies when bound to the metabolite, as known in the art.

Other antibody-based assays could be used, in which the individual antibodies are linked to differently fluorescent beads (e.g. Luminex technology) and so multiplexed assessment of the range of metabolites could be done. Since metabolites are the targets or products of metabolic enzymes, such enzymes may be added to urine samples and the level of activity of the enzyme against the metabolite could be used to quantitate the metabolite. An example of this might be lactate dehydrogenase for the assessment of lactate.

It is envisaged that the present metabolomic approach could be combined with any of the following: CRP; cytokine; and/or autoantibody analyses in tests to predict response. This may be used as part of an individually-tailored therapy, i.e. in personalised medicine. As such, the invention may also provide a method of determining and/or providing a personalised treatment regime to or for a patient, comprising determining the likelihood that the patient, with a disorder treatable with a TNF antagonist, will respond to administration of a TNF-antagonist, the method comprising determining the likelihood of the patient's response to said antagonist based on a metabolic profile of a urine sample from said patient, and including a TNF antagonist in the treatment regime if a sufficiently positive likelihood for a response is determined.

A sufficiently positive likelihood may be 50% or more, but it could be less depending on clinical circumstances, for instance a lower threshold may be acceptable in cases where no other treatments are successful.

In some embodiments, the relationship between baseline metabolite profiles and the change in DAS28 over time (for instance every 3, 6 9 or 12 months) may be assessed to allow progression of the disorder, which is RA, and/or effectiveness of a treatment to be assessed. The assessment may be by known methods such as using PLS-R.

### BRIEF DESCRIPTION OF THE FIGURES

The present invention will now be described with reference to the accompanying figures.

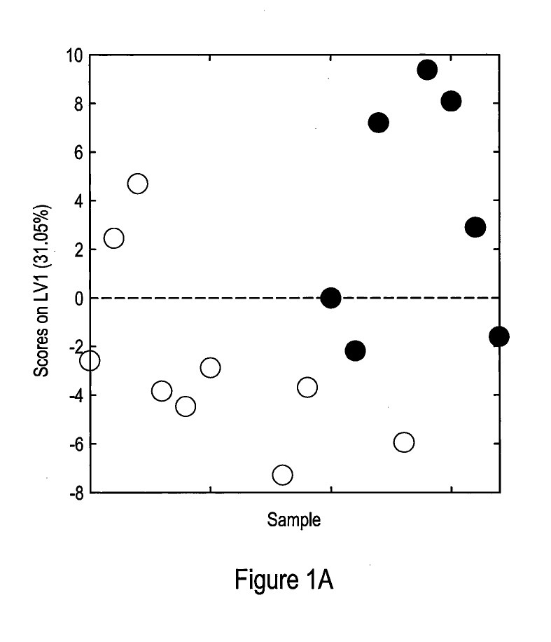

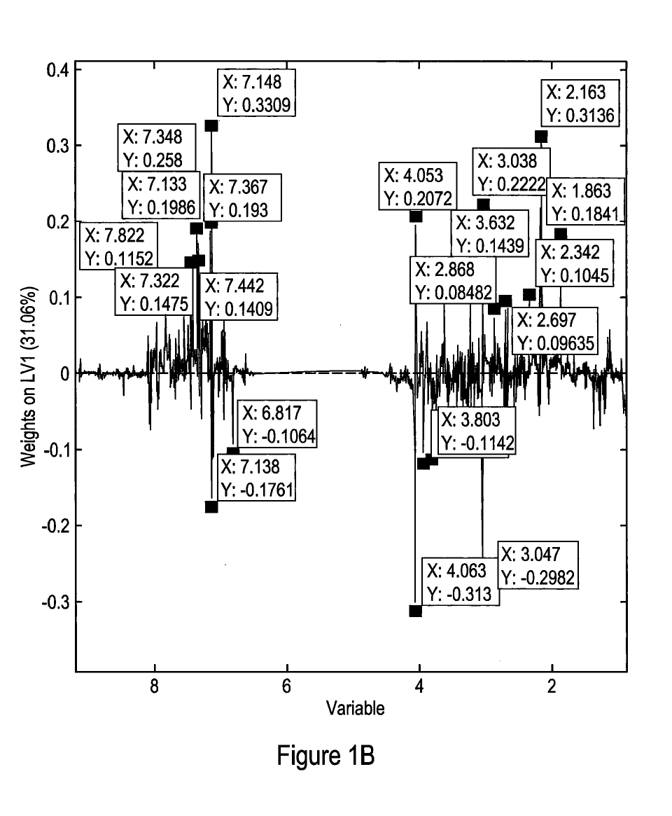

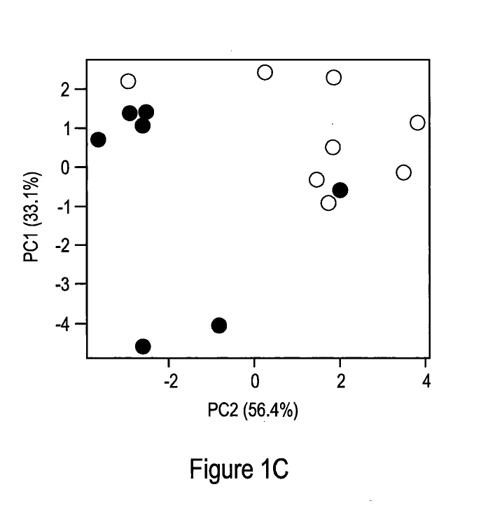

FIG. 1: Metabolic fingerprinting distinguishes between baseline urine samples from RA patients who go on to have good response or not at 12 months.
A. 1D ¹H NMR spectra of baseline urine samples from RA patients who go on to have a good response () or not (◯) to TNF antagonists at 12 months were subjected to supervised analysis (PLS-DA). The values on the axis labels indicate the proportion of the variance captured by each latent variable.
B. Weightings plot of the PLS-DA model of spectral data from baseline urine samples of the RA patients who go on to have good response or not at 12 months highlight major regions of the spectra that distinguish between the sample groups. The values on the x axis indicate chemical shift (ppm) and the values on the y axis indicate the proportion of the variance captured by each latent variable.
C. 1D ¹H NMR spectra of baseline urine from RA patients who go on to have a good response () or not (◯) to TNF antagonists at 12 months were subjected to PCA using GALGO. The values on the axis labels indicate the proportion of the variance captured by each principal component.

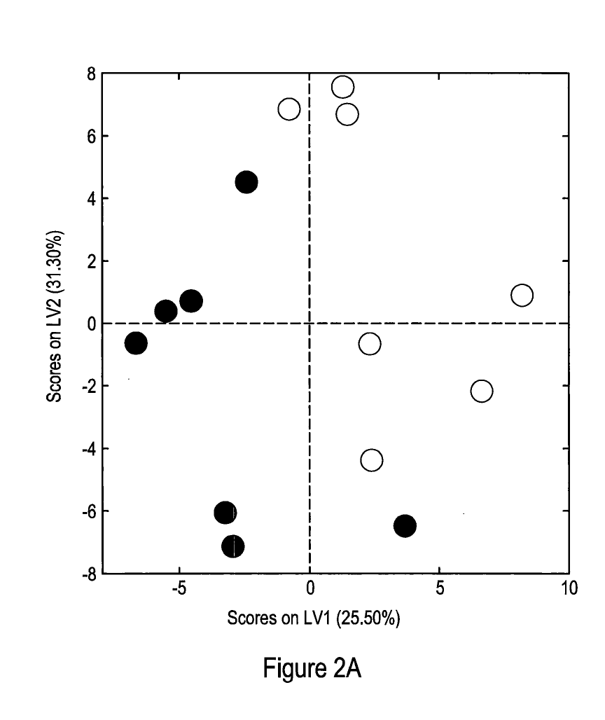

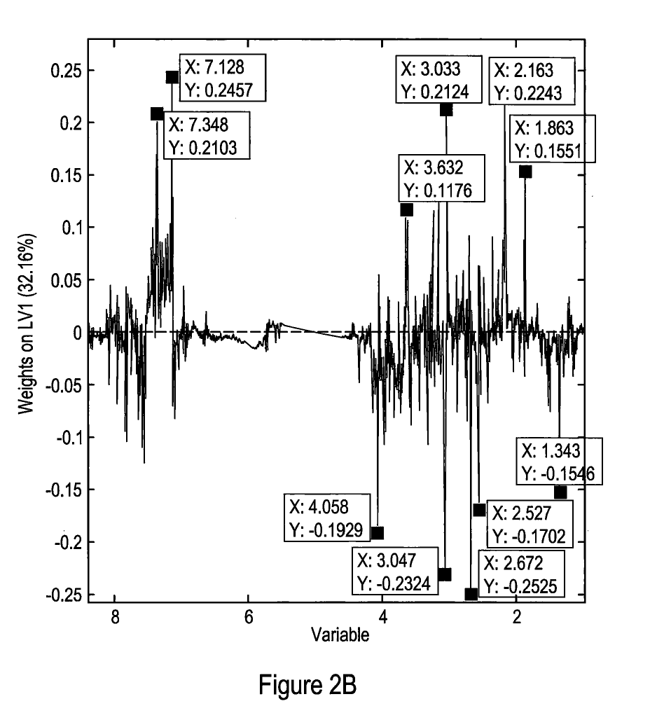

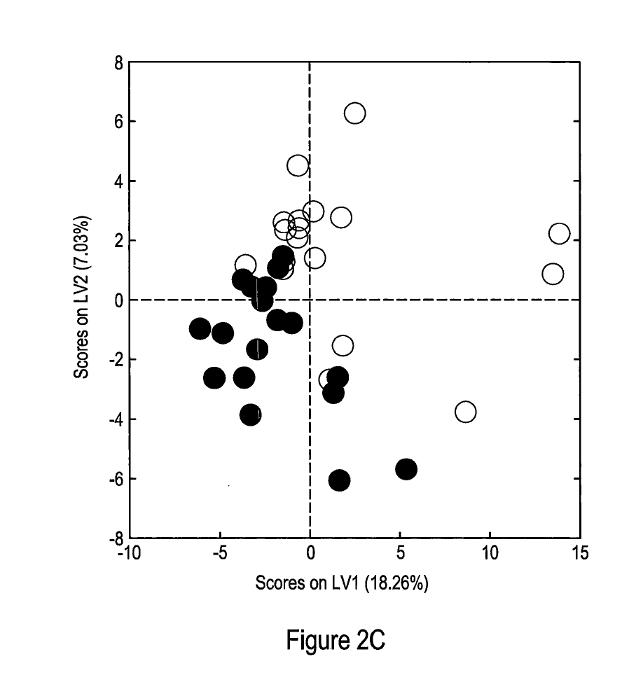

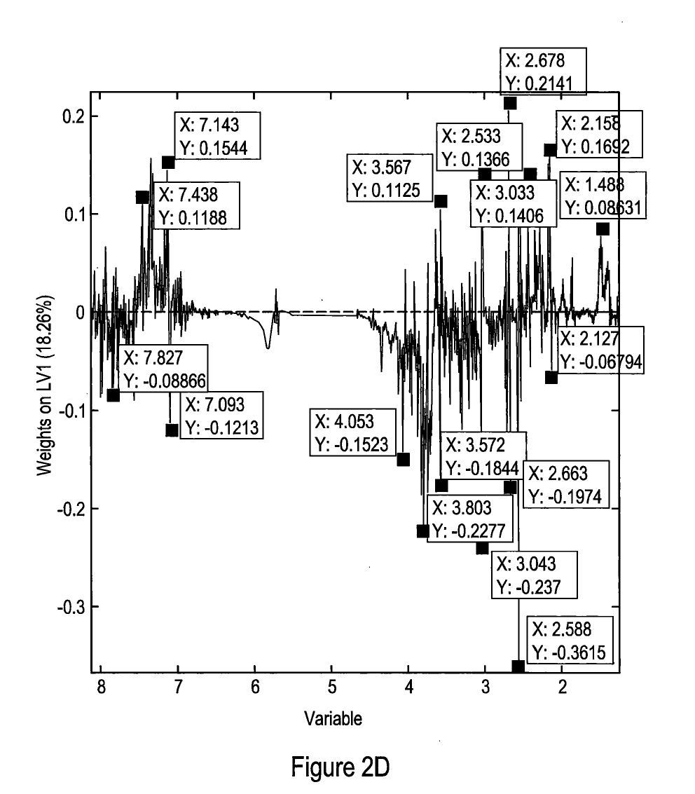

FIG. 2 Metabolic fingerprinting enables identification of metabolites that alter post treatment with TNF antagonists in patients that have a good response.
A. 1D ¹H NMR spectra of urine samples from RA patients at baseline (◯) and 12 weeks () who go on to have a good response to TNF antagonists at 12 months were subjected to supervised analysis (PLS-DA). The values on the axis labels indicate the proportion of the variance captured by each latent variable.
B. Weightings plot of the PLS-DA model of spectral data from urine samples of the patients with RA who responded to TNF antagonists highlights major regions of the spectra that distinguish between the baseline and 12 week samples. The values on the x axis indicate chemical shift (ppm) and the values on the y axis indicate the proportion of the variance captured by each latent variable.
C. 1D ¹H NMR spectra of urine samples from PsA patients at baseline (◯) and 12 weeks () who go on to have a good response to TNF antagonists at 12 months were subjected to supervised analysis (PLS-DA). The values on the axis labels indicate the proportion of the variance captured by each latent variable.
D. Weightings plot of the PLS-DA model of spectral data from urine samples of the patients with PsA who responded to TNF antagonists highlights major regions of the spectra that distinguish between the baseline and 12 week samples. The values on the x axis indicate chemical shift (ppm) and the values on the y axis indicate the proportion of the variance captured by each latent variable.

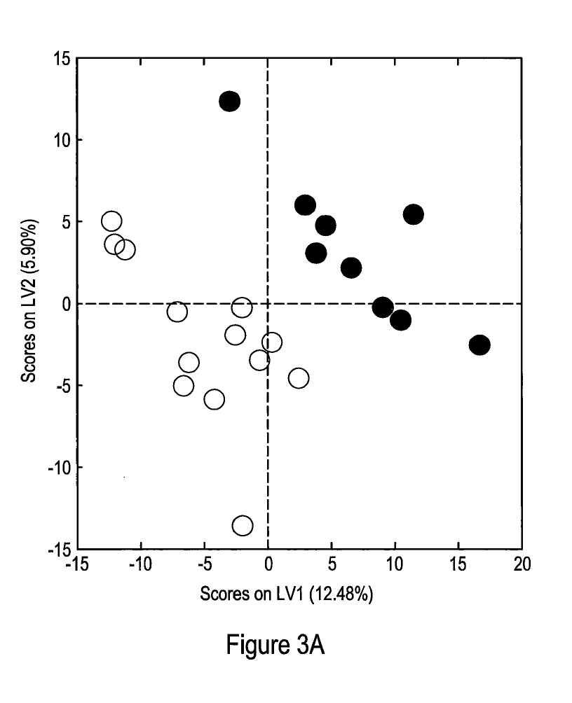

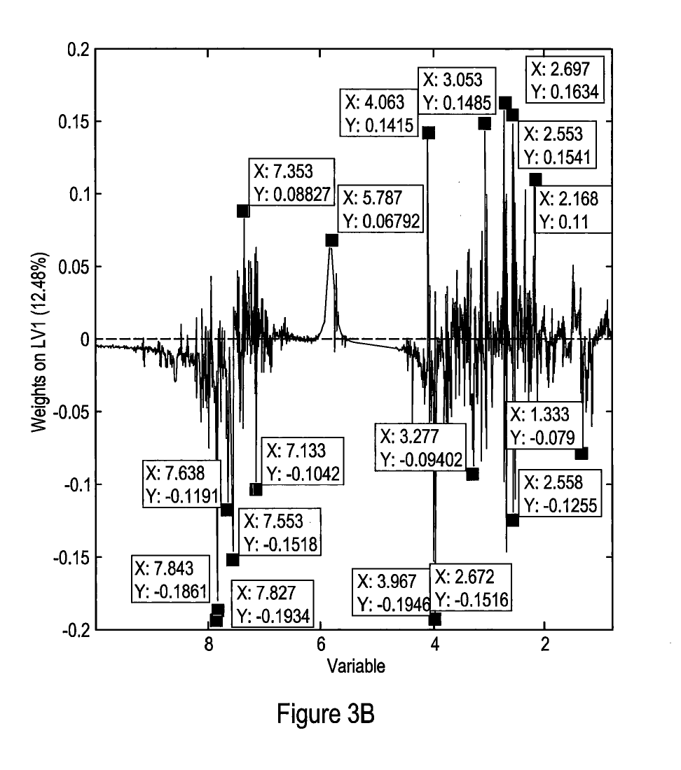

FIG. 3 Metabolic fingerprinting of urines from RA and PsA patients.
A. 1D H NMR spectra of urine samples from RA and PsA patients 12 weeks post treatment with infliximab (◯) and etanercept () who had a good response to treatment were subjected to supervised analysis (PLS-DA). The values on the axis labels indicate the proportion of the variance captured by each latent variable.
B. Weightings plot of the PLS-DA model of spectral data from urine samples of the RA and PsA patients post treatment with infliximab and etanercept who go on to have good response at 12 months highlight major regions of the spectra that distinguish between the sample groups. The values on the x axis indicate chemical shift (ppm) and the values on the y axis indicate the proportion of the variance captured by each latent variable.

### DETAILED DESCRIPTION OF THE INVENTION

Muscle wasting is a common feature of RA and its extent is associated with RA disease activity (8) but low BMI is uncommon, as fat mass is preserved or even increased (9). The extent of the metabolic changes and the types of metabolites seen may therefore be good markers of cytokine mediated inflammatory processes in RA. Several studies have used metabolomic analysis in patients and animal models of inflammatory disease (10-14). Given the integrated nature of systemic metabolism, the analysis of multiple metabolites may provide a better understanding of the disease associated changes. Metabolomic analysis, based on nuclear magnetic resonance (NMR) spectroscopy of biofluids, can be used to identify a broad range of metabolites simultaneously. Using this approach, the identification of several metabolites in cancer and cardiovascular disease has provided insights into disease mechanisms and has highlighted their potential as biomarkers of disease activity and response to therapy (15-17). Systemic changes in many low molecular weight metabolites are reflected by their levels in urine and, indeed, metabolomic analysis of urine samples has been used in inflammatory conditions such as inflammatory bowel disease (IBD) (18-20), to successfully distinguish different types of IBD, and to identify the presence of ongoing intestinal inflammation. Metabolomic profiles have also been shown to alter during therapy (21). However, no one has yet thought to assess whether metabolomic profiles in the urine may have a role in predicting responses to TNF antagonists in patients with RA and PsA.

Anti-TNF therapies are highly effective in rheumatoid (RA) and psoriatic (PsA) arthritis but a significant number of patients exhibit partial or no therapeutic response. Inflammation alters local and systemic metabolism and TNF plays a role in this. We sought to determine if the patient's metabolic fingerprint prior to therapy could predict responses to anti-TNF agents. Urine was collected from 16 RA and 20 PsA patients before and during therapy with infliximab or etanercept. Urine metabolic profiles were assessed using NMR spectroscopy. Discriminating metabolites were identified, and the relationship between metabolic profiles and clinical outcomes was assessed. Baseline urine metabolic profiles discriminated between RA patients who did or did not have a good response to anti-TNF therapy, according to EULAR criteria, with a sensitivity of 88.9% and specificity of 85.7%, with several metabolites (in particular histamine, glutamine, xanthurenic acid and ethanolamine) contributing. There was a correlation between baseline metabolic profiles and the magnitude of change in DAS 28 from baseline to 12 months in RA patients (p=0.04). In both RA and PsA urinary metabolic profiles changed between baseline and 12 weeks of anti-TNF therapy and within the responders, urinary metabolite changes distinguished between etanercept and infliximab treatment. The clear relationship between urine metabolic profiles of RA patients at baseline and their response to anti-TNF therapy may allow development of novel approaches to the optimisation of therapy. Differences in metabolic profiles during treatment with infliximab and etanercept in RA and PsA may reflect distinct mechanisms of action.

In some embodiments, the TNF antagonist is an anti-TNF treatment. This may be Infliximab. In some embodiments, the anti-TNF treatment is Etanercept. In some embodiments, the anti-TNF treatment is infliximab and/or Etanercept. Drugs in this class include etanercept (Enbrel), infliximab (Remicade), adalimumab (Humira), and golimumab (Simponi). Any of these are preferred.

Classification of patients as having a disorder may be by known methods. For example, RA patients may be said to have RA if they meet the criteria of the 1987 American College of Rheumatology classification criteria (22). They may also be positive for rheumatoid factor (RF) and/or anti-CCP antibodies. They may have a disease duration >6 months. They also have a DAS28 score >4.0.

PsA patients may have psoriasis at screening. They may have >3 swollen. They may also have >3 tender peripheral joints. The may have negativity for RF and anti-CCP antibodies and/or a disease duration >6 months. Although this is not believed to be necessary, the patients may have failed treatment with at least one DMARD and may also have been treated with methotrexate. Said treatment with methotrexate may have been at a dose of at least 7.5 mg weekly and this may have been stable for at least 4 weeks prior to commencing anti-TNFα therapy. In some embodiments, no other DMARDs may be allowed within the 4 weeks prior to commencing treatment. In some embodiments, prednisolone may be allowed provided the dose remained stable and did not exceed 10 mg daily.

Treatment regimes will be well known to the skilled person and can in any case be determined according to the standard protocols advised for over the counter or prescription treatments. In some embodiments, these may include infliximab at 3 mg/kg at weeks 0, 2 and 6 and then every 8 weeks until week 46; or etanercept 25 mg twice weekly for 52 weeks. A physician will be able to determine the correct dose based on the latest guidelines for the TNF antagonist. Therapy may be kept stable for the first 3 months. After 3 months, therapy could be changed as required, including escalation of methotrexate therapy to 20 mg weekly.

The conditions in respect of which the present invention may be used in treatment or prophylaxis may be any of the following, which are not mutually exclusive: autoimmune disease associated with joint inflammation; arthritic diseases; and chronic inflammatory diseases, including chronic inflammatory arthritis. Lupus, AS, and/or PsA (Psoriatic arthritis) are preferred and the treatment or prophylaxis of RA is particularly preferred. RA is well-known and described herein, but may be determined according to the 1987 criteria mentioned herein, for instance. Ankylosing spondylitis (AS) is a chronic inflammatory disease and is a form of spondyloarthritis, a chronic, inflammatory arthritis. Psoriatic arthritis is a type of inflammatory arthritis. It develops in up to 30 percent of people who have the chronic skin condition, psoriasis. The condition may also be any condition for which a TNF antagonist may be prescribed, i.e. a condition that responds to a TNF antagonist.

With respect to responsiveness, a good clinical response is of course preferred. This may be defined as a DAS 28<3.2 and/or a DAS 28 improvement>1.2 after therapy (23) in RA. A good response in PsA may be defined as an improvement in 2 factors (with at least one being a joint score) with worsening in none of the following four factors: patient and physician global assessments, tender and swollen joint scores (24). The EULAR criteria mentioned herein are well known and may be used to determine responsiveness if required.

Conditions (disorders) that may be diagnosed may include chronic inflammatory diseases, such as ankylosing spondylitis (AS), RA and/or PsA. RA or PsA are preferred. RA is particularly preferred in some embodiments.

Conditions (disorders) that may be treated may include chronic inflammatory diseases, such as ankylosing spondylitis (AS), RA and/or PsA. RA or PsA are preferred. RA is particularly preferred in some embodiments.

With respect to RA, the urine markers we have found may be indicators of either joint specific degradation processes, or may result from the systemic muscle and tissue changes associated with chronic disease, many of which are mediated through TNFα.

The sample is a urine sample. The urine sample may be collected from a patient, preferably in the morning, and may be snap frozen. It should ideally be stored at −80° C. The sample is preferably collected at baseline, i.e. as soon as the patient presents him or herself, thus creating the initial time point if monitoring is to be conducted. Further samples for monitoring may be collected in the same way every 10-15, ideally 12, weeks. Preferably, monitoring should occur at 3 month, 6 month and/or 12 month intervals (from the initial baseline measurement). Re-assessment after 12 months is particularly preferred.

Measurement of the metabolites may be by standard methods, including HPLC (high performance ion-exchange chromatography), especially for glutamine, and/or fluorometric methods, especially for xanthurenic acid levels.

In some embodiments, the metabolite is histamine. In some embodiments, the metabolites are any one, and ideally all, of: histamine, glutamine, xanthurenic acid and/or ethanolamine. These were identified by all three analytical methods. Furthermore, several metabolites were identified by at least two of the three different methods, including p-hydroxyphenylpyruvic acid, phosphocreatine, thymine, creatinine, phenylacetic acid and xanthine. These findings cross-validate the analyses used. While these individual metabolites (glutamine and xanthurenic acid) contribute strongly to the discrimination, the whole set of metabolites present in the fingerprints is preferred to fully separate the responders from non-responders. Thus, any one, and ideally all, of: p-hydroxyphenylpyruvic acid, phosphocreatine, thymine, creatinine, phenylacetic acid and/or xanthine may also be assayed/profiled. This may be in addition or in place of histamine, glutamine, xanthurenic acid and/or ethanolamine.

In patients with RA who responded to TNF antagonists, there were high levels of glutamine, phenylacetic acid and histamine in the baseline urine samples and higher levels of methylamine and creatinine in the urine post anti-TNF therapy. Similar changes in metabolites were also seen in the urine samples of the patients with PsA who responded to TNF antagonists (FIG. 2). We also found that increases in hippuric acid, citrate and lactic acid were seen with infliximab treatment and increases in choline, phenylacetic acid, urea, creatine and methylamine were seen with etanercept treatment. All three methods identified histamine, glutamine, xanthurenic acid and ethanolamine, while both PLS-DA and PLSR identified creatinine, p-hydroxyphenylpyruvic acid and phosphocreatine and both PLS-DA and GALGO identified phenylacetic acid and xanthine. Histamine, glutamine, phenylacetic acid, xanthine, xanthurenic acid and creatinine were up regulated in the urine samples of the patients that had a good response to therapy whilst ethanolamine, p-hydroxyphenylpyruvic acid and phosphocreatine were down regulated. One metabolite we identified as a strong discriminator in baseline urinary metabolites was histamine. Several of the other metabolites that we have observed were also associated with catabolic processes and tissue degradation for example, glutamine, xanthurenic acid and ethanolamine, can result from tryptophan and other amino acid degradation pathways.

Baseline levels of TNFα may predict the dose of infliximab needed for optimal response (43) and other work has demonstrated that a combination of blood cytokines and autoantibodies can predict responses to etanercept (44). Infliximab and etanercept alter metabolites in the urine differently as there are clear differences in the metabolites at 12 weeks post treatment. Increases in the metabolites hippuric acid, citrate and lactic acid were associated with infliximab treatment and increases in the metabolites choline, phenylacetic acid, urea, creatine and methylamine were associated with etanercept treatment. The presence of choline suggests that etanercept may alter lipid metabolism.

We have also shown that the same metabolites alter in the urines of patients with RA and PsA that responded to TNF antagonists. It may therefore be that chronic inflammatory diseases respond by a common mechanism to TNF antagonists.

### Examples

### Patients and Methods

Patients in Patients were part of a multicentre study (Glasgow Royal Infirmary (PsA patients only),

Queen Elizabeth Hospital, Birmingham (PsA patients only), and Charing Cross Hospital. London (RA patients only)) comparing responses to infliximab and etanercept. All patients were aged 18 or over. RA patients were required to fulfil 1987 American College of Rheumatology classification criteria (22), to be positive for rheumatoid factor (RF) and/or anti-CCP antibodies, have a disease duration >6 months and a DAS28 score >4.0. The PsA patients were required to have psoriasis at screening, >3 swollen and >3 tender peripheral joints, negativity for RF and anti-CCP antibodies and a disease duration >6 months. All patients had failed treatment with at least one DMARD and were treated with methotrexate at a dose of at least 7.5 mg weekly, stable for at least 4 weeks prior to commencing anti-TNFα therapy. No other DMARDs were allowed within the 4 weeks prior to commencing treatment but prednisolone was allowed provided the dose remained stable and did not exceed 10 mg daily.

Participants were randomised to either infliximab 3 mg/kg at weeks 0, 2 and 6 and then every 8 weeks until week 46, or etanercept 25 mg twice weekly for 52 weeks. Therapy was kept stable for the first 3 months. After 3 months, therapy could be changed as required, including escalation of methotrexate therapy to 20 mg weekly in apparent non-responders. Clinical data, including ESR, DAS28 and HAQ scores, were collected at baseline and monthly up to week 52. A good clinical response was defined as a DAS 28<3.2 and a DAS 28 improvement>1.2 after therapy (23) in RA. A good response in PsA was defined as an improvement in 2 factors (with at least one being a joint score) with worsening in none of the following four factors: patient and physician global assessments, tender and swollen joint scores (24). Random urine samples were collected from the patients at baseline and at 12 weeks and were snap frozen and stored at −80° C. The study was conducted in compliance with the Helsinki declaration and ethical approval was obtained from the West Glasgow Ethics Committee. All subjects gave written informed consent.

### Metabolomic Analysis

After thawing, urine samples (1 ml) were centrifuged at 13000×g for 5 mins and samples prepared using a standard protocol (25). Briefly, urine was buffered with phosphate buffer (100 mM), made 10% with D₂O and 0.5 mM with TMSP and the pH adjusted (twice over 30 mins) to pH 7.0. The sample was then centrifuged and loaded into a standard 5 mm NMR tube for spectroscopy.

One-dimensional ¹H spectra were acquired at 300K using a standard spin-echo pulse sequence with water suppression using excitation sculpting on a Bruker DRX 500 MHz NMR spectrometer equipped with a cryoprobe. Samples were processed and data calibrated with respect to the TMSP signal. Spectra were read into Prometab (26) (custom written software in Matlab (version 7, The Math Works, Natick, Mass.)), and were truncated to a 0.8-10.0 ppm (parts per million) range. Spectra were segmented into 0.005 ppm (2.5 Hz) chemical shift ‘bins’ and the spectral areas within each bin were integrated. Spectra were corrected for baseline offset and then normalised to a total spectral area of unity and a generalised log transformation was applied (26). Binned data were then compiled into a matrix, with each row representing an individual sample.

### Statistical Analyses

The data bins from groups of spectra were mean centred and then assessed using the following techniques: (1) Partial least square discriminant analysis (PLS-DA) was used to perform supervised clustering of samples using PLS Toolbox (version 5.8) (Eigenvector Research, Wenatchee, Wash., USA) in Matlab (release 2009a), PLS-DA was cross-validated using Venetian blinds (27), a method which re-assigns randomly selected blocks of data to the PLS-DA model to determine the accuracy of the model in correctly assigning class membership. (2) GALGO, a package available in the statistical environment R, was used to further model the relationship between good responders and those that did not respond well using a genetic algorithm search procedure coupled to statistical modelling methods for supervised classification (28). The results of GALGO analyses are presented as principle component analysis (PCA) plots where the X and Y axes represent first and second principle components providing the greatest variation between samples, and the next largest unrelated variation respectively. GALGO analysis was cross validated using K-fold cross validation where the original sample is randomly partitioned into subsamples and each observation is used for both training and validation. (3) PLS-R, a regression method that identifies which metabolites can predict a continuous variable, was also used. This analysis yields r², a measure of the goodness-of fit of the linear regression, while permutation testing assessed the significance of this prediction.

Lists of metabolites providing the greatest discrimination between groups were then identified for each technique. Using multivariate analyses, peaks with large weightings were identified from the PLS-DA weightings plot. Metabolites were identified using these peaks. GALGO analysis produces a list of ‘bins’ of ranked importance which contribute to the separation between the groups. The PLS-R model represents the 90 “bins” or regions of the spectra which had the greatest influence on the correlation with the change in DAS28. These bins were used to identify the discriminatory metabolites. NMR databases (Human Metabolome Database version 2.5) and Chenomx NMR suite (Chenomx, Alberta, Canada) were used to identify the metabolites.

### Measurement of Metabolites

Glutamine levels were measured in the urine samples using high performance ion-exchange chromatography and xanthurenic acid levels were measured using a fluorometric method (29).

### Results

### Prediction of Response to Anti-TNF Therapy

After 12 months of anti-TNF therapy RA patients were divided into two groups according to their response, as determined by EULAR criteria (Table 1). Response to anti-TNF therapy was also assessed at 3 months but only four patients had a good response (as determined by EULAR criteria) at this stage. For PsA patients only one patient did not respond to treatment with a TNF antagonist according to the predefined response criteria; it was therefore not possible to look at prediction of response in PsA using this particular data set.

NMR spectra of stored baseline urine samples were acquired and analysed in order to identify differences between the two groups as follows:

Supervised PLS-DA analysis (FIG. 1A) showed a clear distinction between patient groups segregated according to clinical response. This model distinguished samples with or without a good response with a sensitivity of 66.7% and a specificity of 57.1%. A weightings plot, which indicates regions of the NMR spectra which contribute to this separation (FIG. 1B), was used to identify the discriminatory metabolites responsible for the difference in response and these are shown in Table 2.

The PLS-R model represents the 90 “bins” or regions of the spectra which had the greatest influence on the correlation with the change in DAS28. The GALGO model identifies the bins which have the greatest influence on the separation. For the PLS-DA model the metabolites were identified from the weightings plot, which indicates regions of the NMR spectra which contribute to the separation. The top 20 bins were identified using GALGO and PLS-R and the metabolites identified from these 20 bins. From the PLS-DA weightings plot the top 20 peaks were identified and the metabolites identified from these.

GALGO analysis was then used to reanalyse the data, firstly in order to verify the results obtained using a further supervised analysis technique, and secondly to utilise the superior modelling power of the GALGO genetic algorithm, which more effectively removes irrelevant variables. The PCA plot yielded by GALGO analysis shows a clear distinction between RA patients segregated according to clinical response (FIG. 1C). The cross validation of this model was shown to distinguish samples from patients who would not have a good response and samples from patients who would have a good response with a greatly improved sensitivity of 88.9% and specificity of 85.7%. GALGO analysis was further used to identify the discriminatory metabolites responsible for the difference in response as shown in Table 2.

Finally, the relationship between baseline metabolite profiles and the change in DAS28 over 12 months was assessed using PLS-R. This analysis was repeated 100 times with and without randomisation of the NMR bin data. There was a significant association between the change in DAS28 and baseline RA metabolites (p=0.04). Permutation testing with 90 NMR bins included (as optimised by forward selection) demonstrated that the regression model was statistically valid (p<0.01). As the National Rheumatoid Arthritis Society of the UK reports, the Disease Activity Score (or DAS 28 assessment) is now recommended at every clinic visit by the British Society for Rheumatology. This measurement is regarded as crucial to guide decisions on starting or altering treatments. DAS 28 as a score is derived from 4 different measurements; the ESR or CRP blood test within the previous 2 weeks, a careful examination of 28 joints for swelling and tenderness and the patients' own assessment of their disease activity and its impact on their health using a visual analogue score. The erythrocyte sedimentation rate (ESR), is the rate at which red blood cells sediment in a period of one hour.

There was a significant difference between the CRP level (C-reactive protein (CRP) is typically found in blood and its levels rise in response to inflammation) of those patients that responded to TNF antagonists compared to those that did not respond (p=0.03). We therefore used PLS-R to further analyse the relationship between CRP and baseline metabolites in order to investigate potential confounding variables; this did not reveal any significant association (p=0.52), suggesting that the difference we have found is independent of the inflammatory processes reflected in the CRP levels. Grouping the metabolite data into quartiles according to the CRP values also failed to separate patient groups on PCA or PLS-3.5 DA (data not shown). Previous studies have shown that patients with RA have subclinical nephropathy (30;31) and that the urinary albumin to creatinine ratio (ACR) is a sensitive marker of disease activity in RA (30). We measured the ACR in the urine samples and there was no significant difference between the ACR of those patients that responded to TNF antagonists compared to those that did not respond (p=0.17) (Table 1). We also performed regression analysis for metabolic profiles at baseline against ACR and this was not significant (p=0.31) suggesting that the relationship we have found between baseline urinary metabolic profiles and DAS28 is independent of micro-albuminuria.

### Comparison of Metabolites Predicting Response to Therapy in RA

Metabolites that associated with a change in DAS28 are shown in Table 2. The metabolites histamine, glutamine, xanthurenic acid and ethanolamine were identified by all three analytical methods. Furthermore, several metabolites were identified by at least two of the three different methods, including p-hydroxyphenylpyruvic acid, phosphocreatine, thymine, creatinine, phenylacetic acid and xanthine. These findings cross-validate the analyses used. We were also able to identify glutamine and xanthurenic acid in the urine samples that were used for NMR analysis using ion-exchange chromatography and a fluorometric method respectively. There was a good correlation between the NMR peaks heights and the assayed levels of xanthurenic acid (p=0.001, r=0.73 using the Spearman correlation test) and a strong trend in the results for the glutamine (p=0.07, r=0.46 using the Spearman correlation test), which help validate our interpretation of the NMR data. However, the assayed levels of glutamine and xanthurenic acid were not significantly higher in the urine samples of the patients who had a good response, which suggests that while these individual metabolites contribute strongly to the discrimination, the whole set of metabolites present in the fingerprints is needed to fully separate the groups.

### Effect of TNFalpha Antagonists on Metabolite Profiles

The details of the patients on etanercept and infliximab are shown in Table 3. We investigated the effect of anti-TNF therapy on metabolic profiles longitudinally, comparing baseline and 12 week (during therapy) urine samples using supervised PLS-DA analysis (sensitivity 71.4% and specificity 57.1% in RA and sensitivity and specificity of 61.1% in PsA) and GALGO (sensitivity 100% and specificity 82.9% in RA and sensitivity 71.8% and specificity 69.5% in PsA). Using the weightings plot we identified that in patients with RA who responded to TNF antagonists, there were high levels of glutamine, phenylacetic acid and histamine in the baseline urine samples and higher levels of methylamine and creatinine in the urine post anti-TNF therapy. Similar changes in metabolites were also seen in the urine samples of the patients with PsA who responded to TNF antagonists (FIG. 2).

Combining RA and PsA patients with a good response, we assessed which urinary metabolites changed after 12 weeks treatment with infliximab and with etanercept using supervised PLS-DA analysis (sensitivity 84.6% and specificity 55.6%) (FIG. 3) and GALGO (sensitivity 86.2% and specificity 100%). Using the weightings plot we found that increases in hippuric acid, citrate and lactic acid were seen with infliximab treatment and increases in choline, phenylacetic acid, urea, creatine and methylamine were seen with etanercept treatment. Due to the small patient numbers, we could not investigate the effects of etanercept and infliximab in RA and PsA separately.

### DISCUSSION

There were clear differences in the metabolic profiles of baseline urine samples of patients with RA who responded well to anti-TNF therapy compared with those who did not. This difference may be important as a novel predictor of responses to TNF antagonists. We have used 3 different data analysis methods to predict response and each found that similar metabolites contributed. We have used GALGO as well as PLS-DA as it has been shown that genetic algorithms optimise the results by removing irrelevant variables and dramatically improve the classification ability of models (32). All three methods identified histamine, glutamine, xanthurenic acid and ethanolamine, while both PLS-DA and PLSR identified creatinine, p-hydroxyphenylpyruvic acid and phosphocreatine and both PLS-DA and GALGO identified phenylacetic acid and xanthine. Histamine, glutamine, phenylacetic acid, xanthine, xanthurenic acid and creatinine were up regulated in the urine samples of the patients that had a good response to therapy whilst ethanolamine, p-hydroxyphenylpyruvic acid and phosphocreatine were down regulated.

One metabolite we identified as a strong discriminator in baseline urinary metabolites was histamine. Urinary histamine metabolites have also been suggested as a marker of disease activity in inflammatory bowel disease (33) suggesting it may be a generic marker of inflammatory processes. Histamine is most obviously associated with mast cell dependent processes such as allergy, and histamine has been identified as a constituent of synovial fluid in arthritis (34). Histological examination of synovial infiltrates in early rheumatoid arthritis has shown mast cells to be present (35), suggesting that these cells could be the source of the discriminating histamine. However, an alternative but significant route for histamine generation is via histidine degradation. Histamine arises in many tissues by the decarboxylation of histidine (36). It has long been known that TNF a promotes cachexia associated with chronic inflammatory disease and this cytokine is known to have direct effects in accelerating muscle breakdown leading to the release of free amino acids including histidine (37). Consistent with this, levels of histidine have been shown to be considerably higher in patients with RA and systemic lupus erythematosus (38) compared to controls. Several of the other metabolites that we have observed were also associated with catabolic processes and tissue degradation for example, glutamine, xanthurenic acid and ethanolamine, can result from tryptophan and other amino acid degradation pathways. Tryptophan has been shown to be down-regulated in plasma of patients with ankylosing spondylitis (AS) compared with to controls (39). The release of tryptophan from its binding serum protein has been shown to correlate with improvement in disease activity in AS (39) and this may be the same in RA. This explains the presence of histamine in general, but what was nevertheless surprising was that the presence of histamine prior to treatment was able to differentiate between responders and non-responders.

A previous metabolomic study has suggested that alterations in serum levels of amino acids may be a useful marker of the presence and severity of osteoarthritis in the knee (40), and the urine markers we have found may be indicators of either joint specific degradation processes, or may result from the systemic muscle and tissue changes associated with chronic disease, many of which are mediated through TNFα.

Previous work has investigated predictors of response to TNFα antagonists. Analysis of patients in the British Society for Rheumatology Biologics Register found that treatment with methotrexate or NSAIDs predicted response to TNF antagonists (41). All the patients in our study were on methotrexate and there were an equal number of patients on NSAIDs who had a good response compared to those who did not. Smoking has been associated with a poor response to infliximab (41) but only one of our patients smoked. Another group has found that the presence of RF or anti-CCP antibodies is associated with a reduced response to TNF antagonists (42) but all of our RA patients were positive for RF and/or anti-CCP antibodies. Baseline levels of TNFα may predict the dose of infliximab needed for optimal response (43) and other work has demonstrated that a combination of blood cytokines and autoantibodies can predict responses to etanercept (44). In our cohort there was a significant difference between the CRP levels in the patients that responded to TNF antagonists compared to those that did not. However, the PLSR analysis failed to find an association between CRP and baseline metabolites suggesting that the association between baseline metabolites and response is independent of CRP.

Infliximab and etanercept alter metabolites in the urine differently as there are clear differences in the metabolites at 12 weeks post treatment. Increases in the metabolites hippuric acid, citrate and lactic acid were associated with infliximab treatment and increases in the metabolites choline, phenylacetic acid, urea, creatine and methylamine were associated with etanercept treatment. The presence of choline suggests that etanercept may alter lipid metabolism.

We have also shown that the same metabolites alter in the urines of patients with RA and PsA that responded to TNF antagonists. It may therefore be that chronic inflammatory diseases respond by a common mechanism to TNF antagonists.

This is the first demonstration that metabolomic techniques using 1D NMR spectra can predict outcome to TNF therapy in patients with severe RA providing a sensitivity and specificity for response that has potential clinical utility. Our present results are robust as they were verified by repeat analysis using alternative statistical techniques. Therefore, although a small initial cohort of patients was used, larger studies should validate these findings.

### DETAILED FIGURE LEGENDS

FIG. 1. Metabolic fingerprinting distinguishes between baseline urine samples from RA patients who go on to have good response or not at 12 months. A. 1D 1H NMR spectra of baseline urine samples from RA patients who go on to have a good response () or not (◯) to TNF antagonists at 12 months were subjected to supervised analysis (PLS-DA). The values on the axis labels indicate the proportion of the variance captured by each latent variable. B. Weightings plot of the PLS-DA model of spectral data from baseline urine samples of the RA patients who go on to have good response or not at 12 months highlight major regions of the spectra that distinguish between the sample groups. The values on the x axis indicate chemical shift (ppm) and the values on the y axis indicate the proportion of the variance captured by each latent variable. C. 1D 1H NMR spectra of baseline urine from RA patients who go on to have a good response () or not (◯) to TNF antagonists at 12 months were subjected to PCA using GALGO. The values on the axis labels indicate the proportion of the variance captured by each principal component.

FIG. 2. Metabolic fingerprinting enables identification of metabolites that after post treatment with TNF antagonists in patients that have a good response. A. 1D 1H NMR spectra of urine samples from RA patients at baseline (◯) and 12 weeks () who go on to have a good response to TNF antagonists at 12 months were subjected to supervised analysis (PLS-DA). The values on the axis labels indicate the proportion of the variance captured by each latent variable. B. Weightings plot of the PLS-DA model of spectral data from urine samples of the patients with RA who responded to TNF antagonists highlights major regions of the spectra that distinguish between the baseline and 12 week samples. The values on the x axis indicate chemical shift (ppm) and the values on the y axis indicate the proportion of the variance captured by each latent variable. C. 1D 1H NMR spectra of urine samples from PsA patients at baseline (◯) and 12 weeks () who go on to have a good response to TNF antagonists at 12 months were subjected to supervised analysis (PLS-DA). The values on the axis labels indicate the proportion of the variance captured by each latent variable, D. Weightings plot of the PLS-DA model of spectral data from urine samples of the patients with PsA who responded to TNF antagonists highlights major regions of the spectra that distinguish between the baseline and 12 week samples. The values on the x axis indicate chemical shift (ppm) and the values on the y axis indicate the proportion of the variance captured by each latent variable.

FIG. 3. Metabolic fingerprinting of urines from RA and PsA patients. A. 1D 1H NMR spectra of baseline urine samples from RA and PsA patients 12 weeks post treatment with infliximab (◯) and etanercept () who had a good response to treatment were subjected to supervised analysis (PLS-DA). The values on the axis labels indicate the proportion of the variance captured by each latent variable. B. Weightings plot of the PLS-DA model of spectral data from urine samples of the RA and PsA patients post treatment with infliximab and etanercept who go on to have good response at 12 months highlight major regions of the spectra that distinguish between the sample groups. The values on the x axis indicate chemical shift (ppm) and the values on the y axis indicate the proportion of the variance captured by each latent variable.

### REFERENCE LIST

(1) Maini R, St Clair E W, Breedveld F, Furst D, Kalden J, Weisman M et al. Infliximab (chimeric anti-tumour necrosis factor alpha monoclonal antibody) versus placebo in rheumatoid arthritis patients receiving concomitant methotrexate: a randomised phase 111 trial. Lancet 1999; 354(9194):1932-9.
(2) Spencer-Green G. Etanercept (Enbrel): update on therapeutic use. Ann Rheum Dis 2000; 59 Suppl 1:i46-i49.
(3) Keystone E C, Kavanaugh A F, Sharp J T, Tannenbaum H, Hua Y, Teoh L S et al. Radiographic, clinical, and functional outcomes of treatment with adalimumab (a human anti-tumor necrosis factor monoclonal antibody) in patients with active rheumatoid arthritis receiving concomitant methotrexate therapy: a randomized, placebo-controlled, 52-week trial. Arthritis Rheum 2004; 50(5):1400-11.
(4) Furst D E, Keystone E C, Braun J, Breedveld F C, Burmester G R, De Benedetti F et al. Updated consensus statement on biological agents for the treatment of rheumatic diseases, 2010. Ann Rheum Dis 2011; 70:12-136,
(5) Bennett A N, Peterson P, Zain A, Grumley J, Panayi G, Kirkham B. Adalimumab in clinical practice. Outcome in 70 rheumatoid arthritis patients, including comparison of patients with and without previous anti-TNF exposure. Rheumatology (Oxford) 2005; 44(8):1026-31,
(6) Bazzani C, Filippini M, Caporali R, Bobbio-Pallavicini F, Favalli E G, Marchesoni A et al. Anti-TNF alpha therapy in a cohort of rheumatoid arthritis patients: Clinical outcomes, Autoimmun Rev 2009; 8(3):260-5.
(7) Rau R. Have traditional DMARDs had their day? Effectiveness of parenteral gold compared to biologic agents. Clin Rheumatol 2005; 24(3):189-202.
(8) Summers G D, Metsios G S, Stavropoulos-Kalinoglou A, Kitas G D. Rheumatoid cachexia and cardiovascular disease. Nat Rev Rheumatol 2010, 6(8):445-51,
(9) Summers G D, Deighton C M, Rennie M J, Booth A H. Rheumatoid cachexia: a clinical perspective. Rheumatology 2008; 47(8):1124-31.
(10) Lin H M, Edmunds S J, Helsby N A, Ferguson L R, Rowan D D. Nontargeted Urinary Metabolite Profiling of a Mouse Model of Crohn's Disease. J Proteome Res 2009; 8(4):2045-57.
(11) Bezabeh T, Somorjai R L, Smith I C P. M R metabolomics of fecal extracts: applications in the study of bowel diseases. Magn Reson Chem 2009; 47:S54-S61.
(12) Marchesi J R, Holmes E, Khan F, Kochhar S, Scanlan P, Shanahan F et al. Rapid and noninvasive metabonomic characterization of inflammatory bowel disease. J Proteome Res 2007; 6(2):546-51.
(13) Young S P, Nessim M, Falciani F, Trevino V, Banerjee S P, Scott R A H at al. Metabolomic analysis of human vitreous humor differentiates ocular inflammatory disease, Mol Vis 2009; 15(125-29):1210-7,
(14) Sinclair A J, Viant M R, Ball A K, Burdon M A, Walker E A, Stewart P M et al. NMR based metabolomic analysis of cerebrospinal fluid and serum in neurological diseases—a diagnostic tool? NMR Biomed 2010; 23(2):123-32.
(15) Pan X Y, Wilson M, Mirbahai L, McConville C, Arvanitis T N, Griffin J L et al. In Vitro Metabonomic Study Detects Increases in UDP-GlcNAc and UDP-GalNAc, as Early Phase Markers of Cisplatin Treatment Response in Brain Tumor Cells. J Proteome Res 2011; 10(8):3493-500.
(16) Sreekumar A, Poisson L M, Rajendiran T M, Khan A P, Cao Q, Vu J D at al. Metabolomic profiles delineate potential role for sarcosine in prostate cancer progression. Nature 2009; 457(7231):910-4,
(17) Brindle J T, Antti H. Holmes E, Tranter G, Nicholson J K, Bethell H W L et at Rapid and noninvasive diagnosis of the presence and severity of coronary heart disease using 1H-NMR-based metabonomics. Nat Med 2002; 8(12):1539,
(18) Schicho R, Nazyrova A, Shaykhutdinov R, Duggan G, Vogel H J, Storr M. Quantitative metabolomic profiling of serum and urine in DSS-induced ulcerative colitis of mice by (1)H NMR spectroscopy. J Proteome Res 2010; 9(12):6265-73.
(19) Murdoch T B, Fu H, MacFarlane S, Sydora B C, Fedorak R N, Slupsky C M. Urinary metabolic profiles of inflammatory bowel disease in interleukin-10 gene-deficient mice, Anal Chem 2008; 80(14):5524-31,
(20) Williams H R T, Cox I J, Walker D G, North B V, Patel V M, Marshall S E at al. Characterization of Inflammatory Bowel Disease With Urinary Metabolic Profiling. Am J Gastroenterol 2009; 104(6):1435-44.
(21) Pan X V, Wilson M, Mirbahai L, McConville C, Arvanitis T N, Griffin J L et al. In Vitro Metabonomic Study Detects Increases in UDP-GlcNAc and UDP-GalNAc, as Early Phase Markers of Cisplatin Treatment Response in Brain Tumor Cells, J Proteome Res 2011; 10(8):3493-500.
(22) Arnett F C, Edworthy S M, Bloch D A, Mcshane D J, Fries J F, Cooper N S at al. The American-Rheumatism-Association 1987 Revised Criteria for the Classification of Rheumatoid-Arthritis. Arthritis Rheum 1988; 31(3):315-24.
(23) van Gestel A M, Haagsma C J, Riel P L C M. Validation of rheumatoid arthritis improvement criteria that include simplified joint counts. Arthritis Rheum 1998; 41(10):1845-50.
(24) Clegg D O, Reda D J, Mejias E, Cannon G W, Weisman M H, Taylor T at al. Comparison of sulfasalazine and placebo in the treatment of psoriatic arthritis—A Department of Veterans Affairs cooperative study. Arthritis Rheum 1996; 39(12):2013-20.
(25) Viant M R, Ludwig C, Rhodes S, Guenther U L, Ailaway D. Validation of a urine metabolome fingerprint in dog for phenotypic classification. Metabolomics 2007; 3(4):453-63.
(26) Viant M R. Improved methods for the acquisition and interpretation of NMR metabolomic data. Biochem Biophys Res Commun 2003: 310(3):943-8,
(27) Chauchard F, Cogdili R, Roussel S, Roger J M, Bellon-Maurel V. Application of LS SVM to non-linear phenomena in NIR spectroscopy: development of a robust and portable sensor for acidity prediction in grapes. Chemometrics and Intelligent Laboratory Systems 2004; 71(2):141-50.
(28) Trevino V, Falciani F. GALGO: an R package for multivariate variable selection using genetic algorithms. Bioinformatics 2006; 22(9):1154-6.
(29) Liu M, Wang G R, Liu T Z, Tsai K J. Improved fluorometric quantification of urinary xanthurenic acid. Olin Chem 1996; 42(3):397-401.
(30) Pedersen L M, Nordin H. Svensson B, Bliddal H. Microalbuminuria in patients with rheumatoid arthritis. Ann Rheum Dis 1995; 54(3):189-92.
(31) Niederstadt C, Rapp T, Tatsis E, Schnabel A, Steinhoff J. Glomerular and tubular proteinuria as markers of nephropathy in rheumatoid arthritis. Rheumatology (Oxford) 1999; 38(1):28-33.
(32) Ramadan Z, Jacobs 0, Grigorov M, Kochhar S. Metabolic profiling using principal component analysis, discriminant partial least squares, and genetic algorithms. Talanta 2006; 68(5):1683-91.
(33) Winterkamp S, Weidenhiller M, Otte P, Stolper J, Schwab D, Hahn E G at al. Urinary excretion of N-methylhistamine as a marker of disease activity in inflammatory bowel disease. Am J Gastroenterol 2002; 97(12):3071-7.
(34) Buckley M G, Walters C, Wong W M, Cawley M I, Ren S, Schwartz L B et al. Mast cell activation in arthritis: detection of alpha- and beta-tryptase, histamine and eosinophil cationic protein in synovial fluid. Clin Sci (Lond) 1997; 93(4):1363-70.
(35) Talc P P, Smeets T J, Daha M R, Kluin P M, Meijers K A, Brand R et at Analysis of the synovial cell infiltrate in early rheumatoid synovial tissue in relation to local disease activity. Arthritis Rheum 1997; 40(2):217-25
(36) Stifel F B, Herman R H. Histidine metabolism. Am J Clin Nutr 1971; 24(2):1207-17.
(37) Goodman M N, Tumor necrosis factor induces skeletal muscle protein breakdown in rats. Am J Physiol 1991; 260(5 Pt 1):E727-E730.
(38) Sitton N G, Dixon J S, Bird H A, Wright V. Serum biochemistry in rheumatoid arthritis, seronegative arthropathies, osteoarthritis, SLE and normal subjects. Br J Rheumatol 1987; 26(2):131-5.
(39) Gao P, Lu C, Zhang F, Sang P, Yang D, Li X et at Integrated G C-M S and L C-M S plasma metabonomics analysis of ankylosing spondylitis. Analyst 2008; 133(9):1214-20.
(40) Zhai G, Wang-Sattler R, Hart D J, Arden N K, Hakim A J, Illig T et al. Serum branched-chain amino acid to histidine ratio: a novel metabolomic biomarker of knee osteoarthritis. Ann Rheum Dis 2010; 69(6):1227-31.
(41) Hyrich K L, Watson K D, Silman A J, Symmons D P. Predictors of response to anti-TNF-alpha therapy among patients with rheumatoid arthritis: results from the British Society for Rheumatology Biologics Register. Rheumatology (Oxford) 2006; 45(12):1558-65.
(42) Potter C, Hyrich K L, Tracey A, Lunt M, Plant D, Symmons D P at al. Association of rheumatoid factor and anti-cyclic citrullinated peptide positivity, but not carriage of shared epitope or PTPN22 susceptibility variants, with anti-tumour necrosis factor response in rheumatoid arthritis. Ann Rheum Dis 2009; 68(1):69-74.
(43) Takeuchi I, Miyasaka N, Tatsuki Y, Yano T, Yoshinari T, Abe T at al. Baseline tumour necrosis factor alpha levels predict the necessity for dose escalation of infliximab therapy in patients with rheumatoid arthritis. Ann Rheum Dis 2011; 70(7):1208-15.
(44) Hueber W, Tomooka B H, Batliwalla F, Li W, Monach P A, Tibshirani R J at al. Blood autoantibody and cytokine profiles predict response to anti-tumor necrosis factor therapy in rheumatoid arthritis. Arthritis Res Ther 2009; 11(3):R76.

1. A method of determining the likelihood that a patient, with a disorder treatable with a TNF antagonist, will respond to administration of a TNF antagonist, the method comprising determining the likelihood of the patient's response to said antagonist based on a metabolic profile of a biological fluid sample from said patient.

2. The method of claim 1, wherein the metabolic profile is determined by assaying for the presence of one or more metabolites in the sample from the patient.

3. The method of claim 1, wherein the sample is a baseline sample taken before treatment with a TNF antagonist has been initiated.

4. The method of claim 1, wherein the sample is serum or urine.

5. The method of claim 2, wherein the metabolite is any metabolite found in the sample and associated with responsiveness of the patient to a TNF antagonist.

6. The method of claim 2, wherein the metabolite is a metabolite associated with tissue degradation or a catabolic process.

7. The method of claim 2, wherein the metabolite is any one or all of the group comprising: histamine; glutamine; xanthurenic acid; and/or ethanolamine.

8. The method of claim 7, wherein the metabolite is histamine.

9. The method of claim 2, wherein the metabolite is any one or all of the group comprising: p-hydroxyphenylpyruvic acid, phosphocreatine, thymine, creatinine, phenylacetic acid and/or xanthine.

10. The method of claim 1, wherein the metabolic profile comprises the presence or absence of the metabolite and it may, optionally, also comprise the levels the metabolite or an indication of whether the metabolite is upregulated or downregulated compared to a reference or threshold value.

11. The method of claim 1, wherein the disorder treatable with a TNF inhibitor is rheumatoid arthritis (RA).

12. The method of claim 1, wherein the disorder treatable with a TNF inhibitor is any one or all of the group comprising: ankylosing spondylitis (AS); Crohn's disease; psoriasis and its associated arthritis (PsA); hidradenitis suppurativa; Behcets Disease; and/or refractory asthma.

13. The method of claim 1, wherein the TNF antagonist is an anti-TNF antibody, including etanercept (Enbrel), infliximab (Remicade), adalimumab (Humira), certolizimumab pegol (Cimzia) and golimumab (Simponi).

14. The method of claim 11, wherein the metabolites are histamine, glutamine, phenylacetic acid, xanthine, xanthurenic acid and creatinine and are upregulated indicating a patient likely to respond to the TNF antagonist.

15. The method of claim 11, wherein the metabolites are ethanolamine, p-hydroxyphenylpyruvic acid and phosphocreatine and are downregulated indicating a patient likely to respond to the TNF antagonist.

16. (canceled)

17. A method for the treatment or prophylaxis of a disorder treatable with a TNF antagonist, comprising: identifying that the patient will respond to administration of a TNF-antagonist by determining the likelihood of the patient's response to said antagonist based on a metabolic profile of a biological fluid sample from said patient; and administering a suitable treatment to said patient.

18. A kit for determining the metabolic profile of a biological fluid sample comprising: means for collecting and/or testing samples and the metabolites therein; and a set of at least one control metabolite corresponding to the metabolites being tested for in the sample from a patient.
